(19) United States
(12) Patent Application Publication
(10) Pub. No.: US 2023/0301594 A1
(43) Pub. Date: Sep. 28, 2023

(54) DETECTING DEVICE AND MEASURING APPARATUS

(71) Applicant: Seiko Epson Corporation, Tokyo (JP)

(72) Inventors: Takefumi FUKAGAWA, Fujimi-Machi Suwa-Gun (JP); Hiromu TAKAYAMA, Chino-Shi (JP); Tetsuya YAMAMOTO, Suwa-Shi (JP); Takehiko KUBOTA, Matsumoto-Shi (JP)

(73) Assignee: Seiko Epson Corporation, Tokyo (JP)

(21) Appl. No.: 18/182,363

(22) Filed: Mar. 13, 2023

(30) Foreign Application Priority Data
Mar. 14, 2022 (JP) 2022-038823

(51) Int. Cl. A61B 5/00; G01D 5/24

(52) U.S. Cl. CPC A61B 5/6844; A61B 5/681; G01D 5/24

(57) ABSTRACT

A detecting device includes a substrate and a plurality of detecting elements arranged in a matrix at the substrate. Each of the plurality of detecting elements includes a light emitting portion that emits light toward a living body, a light receiving portion that can receive light incident from the living body based on the light emitted from the light emitting portion, and a contact detecting portion that detects contact with the living body. The light emitting portion in each of the plurality of detecting elements emits light when the contact detecting portion in the same detecting element has detected contact with the living body.

The present application is based on, and claims priority from JP Application Serial Number 2022-038823, filed Mar. 14, 2022, the disclosure of which is hereby incorporated by reference herein in its entirety.

### BACKGROUND

### 1. Technical Field

The present disclosure relates to a detecting device and a measuring apparatus including a light emitting portion and a light receiving portion.

### 2. Related Art

Various measurement techniques for non-invasively measuring biological information such as a heartbeat have been proposed. JP-A-2018-061675 and JP-A-2003-070757 describe a detecting device including a light emitting portion that emits light toward a living body and a light receiving portion that receives light reflected by the living body. Biological information can be obtained by analyzing a signal output from the light receiving portion in this type of detecting device.

The detecting device of JP-A-2018-061675 includes a substrate, on whose surface a light emitting element and a light receiving element are arranged, and a shielding member that covers the substrate. The shielding member includes a reflecting portion that reflects light from the light emitting element toward the living body and a light-blocking portion positioned between the light emitting element and the light receiving element. Installing the light-blocking member increases the light utilization efficiency of the light emitting portion while dealing with stray light for the light receiving portion.

The detecting device (pulse meter) of JP-A-2003-070757 includes a contact detecting circuit for detecting whether or not it is in contact with the user’s skin. The contact detecting circuit includes an electrode surrounding a light emitting portion (a light emitting diode) and a light receiving portion (a photodiode). The detecting device performs a process of turning off the driving of the pulse meter when the contact detecting circuit has determined that the pulse meter has not been in contact with the user’s skin for one second or longer.

Although the detecting device of JP-A-2018-061675 is designed to improve the light utilization rate and achieve power saving, it requires a space for providing a light-blocking wall and it is difficult to achieve both size reduction and power saving.

Although the detecting device (pulse meter) of JP-A-2003-070757 can achieve power saving by detecting bad wearing and switching it on or off, the entire device will be turned off, disabling measurement, unless the peripheries of the light emitting portion and the light receiving portion are brought into contact with the skin.

### SUMMARY

To solve the above problems, a detecting device of the present disclosure includes a substrate and a plurality of detecting elements arranged in a matrix at the substrate, wherein each of the plurality of detecting elements includes a light emitting portion configured to emit light toward a living body, a light receiving portion configured to receive light incident from the living body based on the light emitted from the light emitting portion, and a contact detecting portion configured to detect a contact with the living body, and the light emitting portion in each of the plurality of detecting elements is configured to emit light based on the detection of the contact with the living body by the contact detecting portion in the same detecting element.

A measuring apparatus of the present disclosure includes the above detecting device and an information analysis unit configured to identify biological information from a detection signal indicating a detection result of the detecting device.

### BRIEF DESCRIPTION OF THE DRAWINGS

FIG. 1 is a side view of a measuring device to which the present disclosure is applied.

FIG. 2 is a block diagram showing functional components of the measuring device to which the present disclosure is applied.

FIG. 3 is a plan view of a substrate on which detecting elements are arranged.

FIG. 4 is a plan view of detecting elements.

FIG. 5 is a circuit diagram of a detecting element.

FIG. 6 is a cross-sectional view schematically showing cross-sectional structures of a light receiving portion and a light emitting portion.

FIG. 7 is a cross-sectional view schematically showing cross-sectional structures of the light receiving portion and a contact detecting portion.

FIG. 8 is a timing chart of one detection cycle of the detecting element.

FIG. 9 is a diagram schematically showing an operating state of the detecting device.

### DESCRIPTION OF EXEMPLARY EMBODIMENTS

Hereinafter, embodiments of the present disclosure will be described with reference to the drawings. In each drawing below, each member is schematically shown as having a recognizable size and actual dimensions and ratios thereof may differ from those shown in the drawings.

### Measuring Apparatus

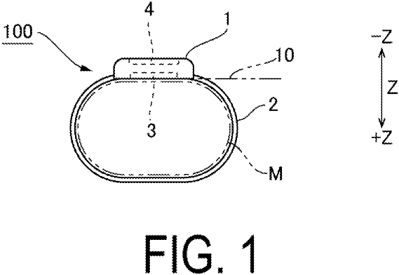

FIG. 1 is a side view of a measuring apparatus 100 to which the present disclosure is applied. The measuring apparatus 100 is a biometric device that non-invasively measures biological information. The measuring apparatus 100 is used oriented toward a site (hereinafter referred to as a “measuring site M”) that is a measurement target on a body (a living body) of a subject (that is, a user of the measuring apparatus 100). In an example shown in FIG. 1, the measuring apparatus 100 is a wristwatch-type portable device that includes a housing 1 and a belt 2. The measuring site M is a wrist of the subject. The measuring apparatus 100 is used worn on the wrist (the measuring site M) of the subject by winding the belt 2 around the wrist with a detecting surface 10 of the housing 1 facing a skin surface of the wrist.

In the present specification, an X direction, a Y direction, and a Z direction are directions orthogonal to each other. The Z direction is a direction normal to the detecting surface 10. A +Z direction is a direction from the detecting surface 10 to the measuring site M and a -Z direction is a direction from the measuring site M to the detecting surface 10.

In the present specification, biological information is exemplified by a heartbeat (for example, a pulse rate) and an oxygen saturation (SpO₂) of the subject. The heartbeat indicates the change of the internal volume of a blood vessel over time due to the pulsation of the heart. The oxygen saturation indicates the proportion (%) of hemoglobin bound to oxygen in hemoglobin in the blood of the subject and is an index for evaluating the respiratory function of the subject.

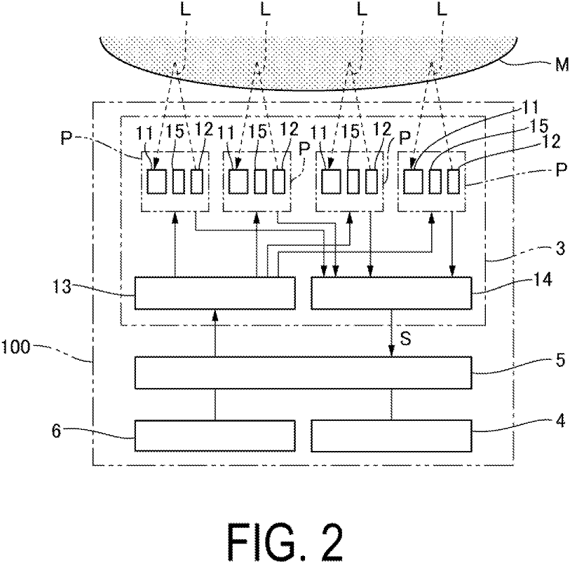

FIG. 2 is a block diagram showing functional components of the measuring apparatus 100 to which the present disclosure is applied. As shown in FIG. 2, the measuring apparatus 100 includes a control device 5, a storage device 6, a display device 4, and a detecting device 3. The control device 5 and the storage device 6 are disposed inside the housing 1. The detecting device 3 is disposed on the detecting surface 10. The display device 4 is disposed on a surface of the housing 1 opposite to the detecting surface 10. The display device 4 displays various images including measurement results under the control of the control device 5. The display device 4 is, for example, a liquid crystal display panel.

In addition to the functional components shown in FIG. 2, the measuring apparatus 100 may include an operation unit such as an operation button or a touch panel disposed on the surface of the housing 1 and may be configured to input an operation signal corresponding to an operation on the operation unit to the control device 5. The measuring apparatus 100 may also include a communication unit for outputting measurement results to the outside and inputting a signal from the outside to the control device 5. Alternatively, the measuring apparatus 100 may include an audio output unit or a vibrating unit as means for notifying of measurement results.

As shown in FIG. 2, the detecting device 3 includes light receiving portions 11, light emitting portions 12, a drive circuit 13, an output circuit 14, and contact detecting portions 15. A part or whole of the drive circuit 13 and the output circuit 14 can be installed as circuits external to the detecting device 3. That is, the drive circuit 13 and the output circuit 14 may be omitted from the detecting device 3.

The detecting device 3 is a reflective optical sensor module that emits light through the detecting surface 10 and receives light that has been incident on the detecting surface 10 from the measuring site M to generate a detection signal S. Light emitted from a light emitting portion 12 is incident on the measuring site M through the detecting surface 10 and propagates in the measuring site M while being repeatedly reflected and scattered and is then emitted from the measuring site M and incident on a light receiving portion 11 disposed on the detecting surface 10. The detecting device 3 detects that the living body (the measuring site M) is in contact with the detecting surface 10 through the contact detecting portions 15 and switches the light emitting portions 12 and the light receiving portions 11 on and off. Thus, the light receiving portions 11, the light emitting portions 12, and the contact detecting portions 15 are arranged on the detecting surface 10.

The detecting device 3 includes a plurality of detecting elements P each including one light receiving portion 11, one light emitting portion 12, and one contact detecting portion 15. Upon detecting contact with the living body (measuring site M) through a contact detecting portion 15, each detecting element P emits light through a light emitting portion 12 provided in the same detecting element P as that including the contact detecting portion 15 which has output a contact detection signal and outputs a detection signal corresponding to the intensity of light received by a light receiving portion 11 provided in the same detecting element P as that including the light emitting portion 12 that has emitted light.

The drive circuit 13 supplies a drive current to the light emitting portion 12 of each detecting element P to cause it to emit light. Light L emitted from the light emitting portion 12 of each detecting element P is, for example, green light having a green wavelength band of 520 nm to 550 nm and a peak wavelength of 520 nm. The wavelength of light emitted from the light emitting portion 12 is not limited to that of this wavelength band.

The light receiving portion 11 of each detecting element P receives light L that has been emitted from the light emitting portion 12 in the same detecting element P and then returned to the detecting surface 10 after being repeatedly reflected and scattered in the measuring site M. The light receiving portion 11 generates a detection signal corresponding to the intensity of the received light. The output circuit 14 is configured to include, for example, an A/D converter that converts the detection signal generated by the light receiving portion 11 from analog to digital and an amplifier circuit that amplifies the converted detection signal (both not shown) and generates a detection signal S representing the intensity of light received by the light receiving portion 11.

Each detection signal S is a heartbeat signal including periodic fluctuations corresponding to pulsations (volume heartbeats) of the artery inside the measuring site M because the amounts of light absorbed by blood during dilation and contraction of blood vessels generally differ.

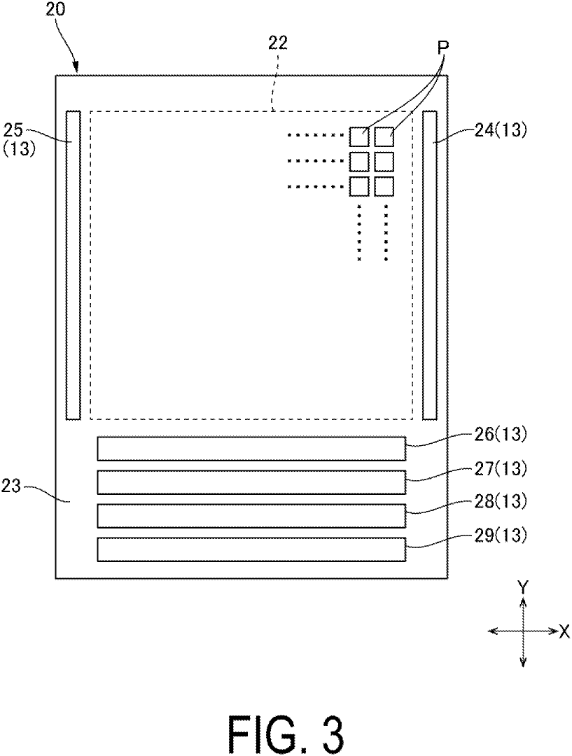

The drive circuit 13 and the output circuit 14 are mounted, for example, on a substrate in the form of an IC chip. In the first embodiment, the drive circuit 13 is mounted on a substrate 20 (see FIG. 3) on which the plurality of detecting elements P are formed as will be described later. The output circuit 14 may be mounted on the substrate 20 on which the detecting elements P are formed together with the drive circuit 13 or may be mounted on a separate substrate. Alternatively, the output circuit 14 may be installed as a circuit external to the detecting device 3.

The control device 5 is an arithmetic processing unit such as a central processing unit (CPU) or a field-programmable gate array (FPGA) and controls the entirety of the measuring apparatus 100. The storage device 6 includes, for example, a non-volatile semiconductor memory and stores a program executed by the control device 5 and various data used by the control device 5. It is also possible to adopt a configuration in which the functions of the control device 5 are distributed over a plurality of integrated circuits or a configuration in which some or all of the functions of the control device 5 are realized by a dedicated electronic circuit. Although the control device 5 and the storage device 6 are shown as separate elements in FIG. 2, a control device 5 including the storage device 6 can also be realized, for example, by an ASIC.

The control device 5 identifies biological information of the subject from the detection signal S generated by the detecting device 3 by executing the program stored in the storage device 6. The control device 5 identifies a heartbeat of the subject from the detection signal S representing the intensity of light received by the light receiving portion 11. The control device 5 can identify, for example, the pulse rate of the subject based on the detection signal S.

The control device 5 functions as an information analysis unit that identifies biological information from the detection signal S indicating the detection result of the detecting device 3 as described above. The control device (information analysis unit) 5 causes the display device 4 to display the biological information identified from the detection signal S. It is also possible to notify the user of the measurement result by voice output. It is also possible to notify the user of a warning (possibility of impaired physical function) when the measurement result has fluctuated to values outside of a predetermined range.

### Planar Structure of Detecting Elements

FIG. 3 is a plan view of the substrate 20 on which detecting elements P are arranged. The detecting device 3 includes the substrate 20 and a plurality of detecting elements P formed at the substrate 20. The substrate 20 has a rectangular first region 22 and a rectangular frame-shaped second region 23 surrounding the first region 22.

The plurality of detecting elements P are arranged in a matrix in the X and Y directions in the first region 22. The drive circuit 13 for driving each detecting element P is disposed in the second region 23. The drive circuit 13 includes a first scan line drive circuit 24 and a second scan line drive circuit 25 that extend in the Y direction, a first signal line drive circuit 26, a second signal line drive circuit 27, and a third signal line drive circuit 28 that extend in the X direction, and a terminal portion 29. The first scan line drive circuit 24 and the second scan line drive circuit 25 are disposed at positions that sandwich the first region 22 in the X direction. The terminal portion 29 is formed at a position in the Y direction opposite to the first region 22 across the first signal line drive circuit 26, the second signal line drive circuit 27, and the third signal line drive circuit 28. The substrate 20 is electrically coupled to an external circuit such as the control device 5 through the terminal portion 29.

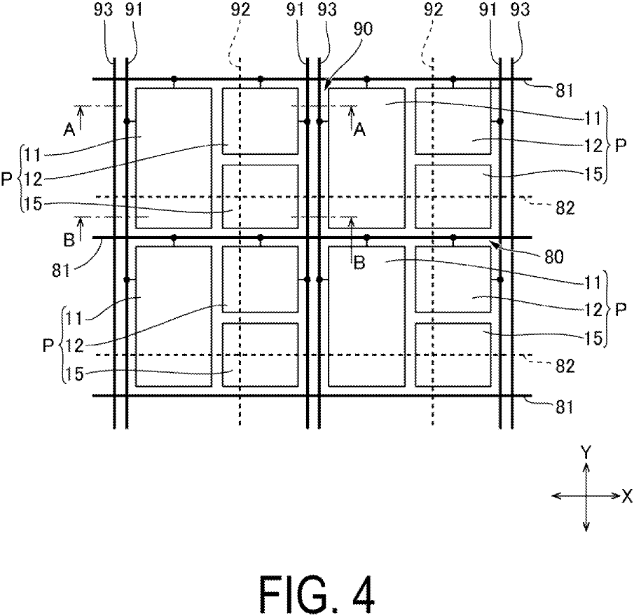

FIG. 4 is a plan view of detecting elements P, which is a partially enlarged view of the first region 22. A plurality of first scan lines 81 extending in the X direction and a plurality of first signal lines 91 extending in the Y direction are formed in the first region 22. One detecting element P is formed in each grid cell formed by the intersection of the first scan lines 81 and the first signal lines 91. Each detecting element P includes one light receiving portion 11, one light emitting portion 12, and one contact detecting portion 15.

A second scan line 82 extending in the X direction, a second signal line 92 extending in the Y direction, and a third signal line 93 extending in the Y direction are also formed in the first region 22 for each detecting element P. The first scan line 81 is formed in an inter-element wiring region 80 provided in each gap between detecting elements P adjacent to each other in the Y direction in the first region 22. The second scan line 82 is formed in a region that overlaps the contact detecting portion 15 of each detecting element P in the Z direction. A first signal line 91 and a third signal line 93 are also formed in an inter-element wiring region 90 provided in each gap between detecting elements P adjacent to each other in the X direction. The second signal line 92 is formed in a region that overlaps the contact detecting portion 15 of each detecting element P in the Z direction.

The arrangements of signal lines and scan lines are not limited to the above arrangements. For example, the second scan lines 82 can be formed in the inter-element wiring regions 80 and the second signal lines 92 can be formed in the inter-element wiring regions 90.

In the present embodiment, the light emitting portion 12 and the contact detecting portion 15 are arranged in the Y direction as shown in FIG. 4. The light receiving portion 11 has a rectangular shape elongated in the Y direction and is aligned with the light emitting portion 12 and the contact detecting portion 15 in the X direction. Thus, the light receiving portion 11 and the light emitting portion 12 are formed at positions adjacent to each other in the X direction on a first surface 21 and the light receiving portion 11 has a larger area than the light emitting portion 12.

### Circuit Configuration of Detecting Element

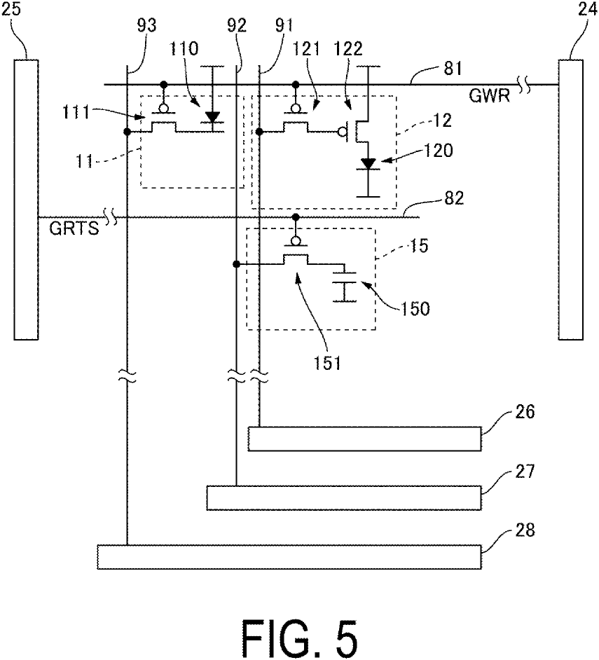

FIG. 5 is a circuit diagram of a detecting element P. The light emitting portion 12 includes an organic EL light emitting element 120, a first switching transistor 121, and a drive transistor 122. The first switching transistor 121 has a gate electrode coupled to the first scan line 81 and source and drain electrodes, one of which is coupled to the first signal line 91. The source and drain electrodes of the drive transistor 122 are coupled to power lines (anode and cathode wirings) of the organic EL light emitting element 120 such that they are in series with the organic EL light emitting element 120. The other of the source and drain electrodes of the first switching transistor 121 is coupled to a gate electrode of the drive transistor 122.

The first scan line 81 is coupled to the first scan line drive circuit 24. The first signal line 91 is coupled to the first signal line drive circuit 26. The first scan line 81 and the first signal line 91 are control lines for controlling on/off of the first switching transistor 121 which is at the intersection of the first signal line 91 and the first scan line 81. The first scan line drive circuit 24 and the first signal line drive circuit 26 select the first scan line 81 and the first signal line 91 to which a potential is to be supplied based on the contact detection signal from the contact detecting portion 15 and switches the first switching transistor 121 on and off. While the first switching transistor 121 is on, power is supplied to the organic EL light emitting element 120 via the drive transistor 122 and thus the light emitting portion 12 emits light.

The contact detecting portion 15 is a capacitive touch sensor. The contact detecting portion 15 includes a capacitance detecting portion 150, in which the capacitance between a pair of detecting electrodes changes according to its proximity to an object (a conductor) from the outside, and a second switching transistor 151. The second switching transistor 151 has a gate electrode coupled to the second scan line 82 and source and drain electrodes coupled to the second signal line 92. One of the source and drain electrodes of the second switching transistor 151 is coupled to one of the pair of detecting electrodes of the capacitance detecting portion 150.

The second scan line 82 is coupled to the second scan line drive circuit 25. The second scan line 82 is a control line for controlling on/off of the second switching transistor 151. The second signal line 92 is coupled to the second signal line drive circuit 27. The second signal line drive circuit 27 is a contact detection signal read circuit that outputs a contact detection signal via the second signal line 92. While the second switching transistor 151 is on, the contact detecting portion 15 outputs a contact detection signal corresponding to a change in the capacitance of the capacitance detecting portion 150 via the second signal line 92.

The light receiving portion 11 includes a photodiode 110 and a third switching transistor 111 which is coupled to power lines (anode and cathode wirings) of the photodiode 110. The third switching transistor 111 has a gate electrode coupled to the first scan line 81 and source and drain electrodes coupled to the third signal line 93 and the photodiode 110. The third signal line 93 is coupled to the third signal line drive circuit 28.

The first scan line 81 is a control line for controlling on/off of the third switching transistor 111. That is, in the present embodiment, the light receiving portion 11 and the light emitting portion 12 in the same detecting element P are coupled to the first scan line 81 which is a common control line. Thus, the light receiving portion 11 and the light emitting portion 12 in the same detecting element P are controlled such that they are synchronously turned on and off. The light receiving portion 11 outputs a detection signal corresponding to its intensity of received light via the third signal line 93 when the light emitting portion 12 arranged in the same detecting element P has emitted light.

### Cross-sectional Structure of Detecting Element

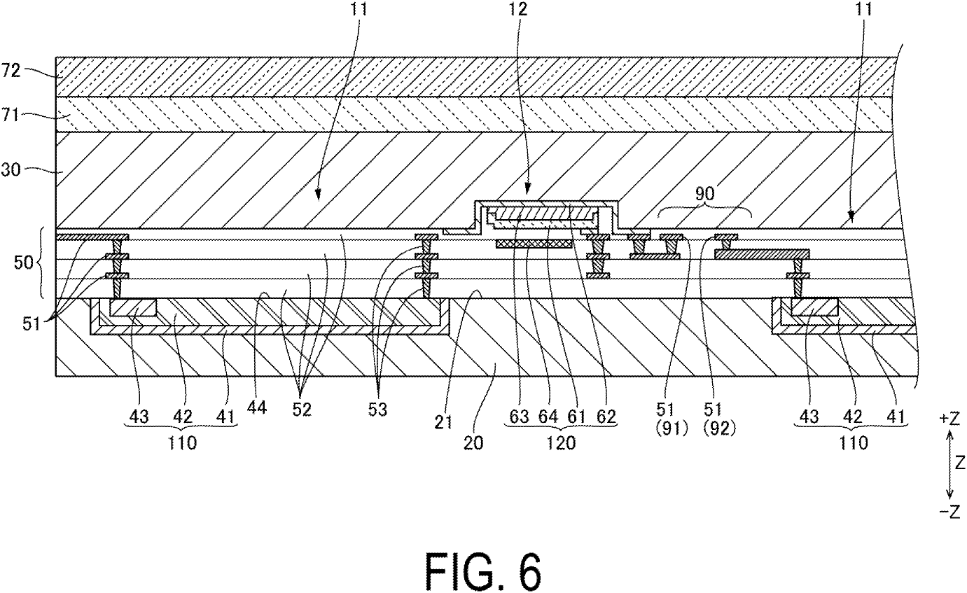

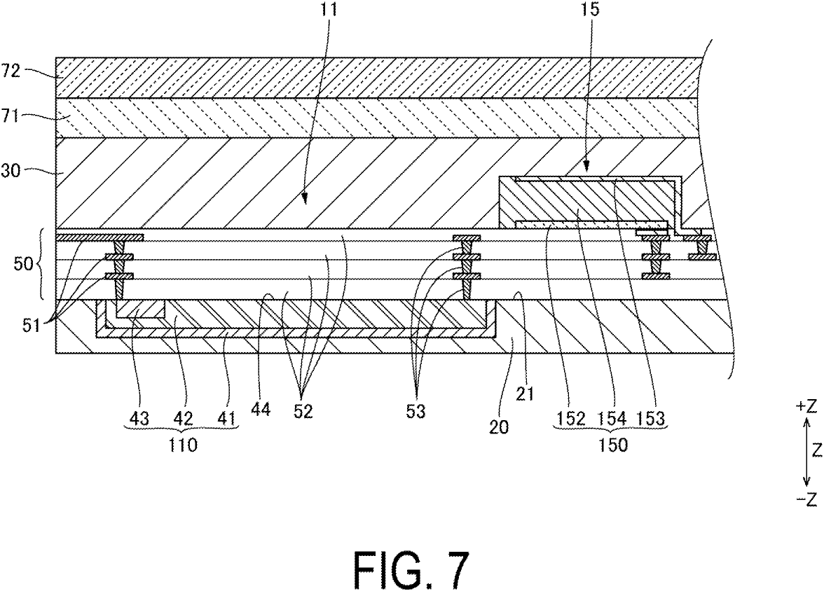

FIGS. 6 and 7 are cross-sectional views schematically showing a cross-sectional structure of a detecting element P. FIG. 6 is a cross-sectional view schematically showing cross-sectional structures of the light receiving portion 11 and the light emitting portion 12, taken along line A-A of FIG. 4. FIG. 7 is a cross-sectional view schematically showing cross-sectional structures of the light receiving portion 11 and the contact detecting portion 15, taken along line B-B of FIG. 4. As shown in FIGS. 6 and 7, a direction normal to the substrate 20 coincides with the Z direction (the direction normal to the detecting surface 10). The light receiving portion 11, the light emitting portion 12, and the contact detecting portion 15 are formed at the first surface 21 of the substrate 20. A first surface 21 is a surface facing in the +Z direction.

The substrate 20 is made of a semiconductor material such as silicon (Si) and is used as a base (a substrate) on which the photodiode 110, the organic EL light emitting element 120, and the capacitance detecting portion 150 are formed. In FIGS. 6 and 7, the switching transistors provided in the light receiving portion 11, the light emitting portion 12, and the contact detecting portion 15 are not shown.

As shown in FIGS. 6 and 7, the photodiode 110 includes a photoelectric conversion portion embedded in a surface layer of the substrate 20 on the first surface 21 side. The photoelectric conversion portion includes an n-type semiconductor layer 41 embedded in the substrate 20, a p-type semiconductor layer 42 formed inside the n-type semiconductor layer 41, and an n-type semiconductor layer 43 buried inside the p-type semiconductor layer 42. The p-type semiconductor layer 42 is exposed on the first surface 21 of the substrate 20. The p-type semiconductor layer 42 forms a light receiving surface 44 on which light that has passed through wiring layers 50 formed at the first surface 21 is incident.

The stack of wiring layers 50 includes conductive layers 51 made of a light-reflecting material such as an aluminum-copper alloy (AlCu) or titanium nitride (TIN), interlayer insulating films 52 made of a light-transmitting material such as silicon dioxide (SiO₂) or silicon nitride (SiN), and conductive plugs 53 formed in the interlayer insulating film 52. The conductive plugs 53 are made of, for example, a light-absorbing material such as W (tungsten). The stack of wiring layers 50 is formed at the substrate 20 continuously over the forming area of the detecting element P and the inter-element wiring regions 80 and 90.

In a region of the stack of wiring layers 50 which overlaps the photodiode 110 on the +Z side thereof, the conductive plugs 53 and the conductive layers 51 form wirings electrically coupled to an anode and a cathode (not shown) of the photodiode 110. The wirings coupled to the anode and cathode of the photodiode 110 extend to the inter-element wiring regions 80 and 90 in the stack of wiring layers 50.

As shown in FIG. 6, the organic EL light emitting element 120 includes a first electrode 61 and a second electrode 62 that face each other in the Z direction and an organic light emitting layer 63 formed between the first electrode 61 and the second electrode 62. The organic light emitting layer 63 contains an organic material that emits light when supplied with a current. For example, the organic light emitting layer 63 contains a green light emitting material and light L emitted from the light emitting portion 12 is green light. The organic EL light emitting element 120 also includes a reflective layer 64 formed in the stack of wiring layers 50 interposed between the first electrode 61 and the substrate 20. The reflective layer 64 is formed at a position overlapping the organic light emitting layer 63 on the -Z direction side (that is, on the substrate 20 side) thereof. The reflective layer 64 is made of, for example, a light-reflecting material such as an aluminum copper alloy (AlCu).

The organic EL light emitting element 120 is a top emission type organic EL element capable of extracting light from the +Z side of the organic light emitting layer 63. The first electrode 61 is formed at the surface of the stack of wiring layers 50. The first electrode 61 is an anode. The first electrode 61 is a transparent electrode made of, for example, indium tin oxide (ITO). The second electrode 62 faces the first electrode 61 from the +Z direction. The second electrode 62 is a cathode. The second electrode 62 is, for example, a light-transmitting electrode made of a silver-magnesium alloy (AgMg). A light-blocking electrode may be used as the first electrode 61 of the organic EL light emitting element 120. In addition to the organic light emitting layer 63, a hole injection layer, a hole transport layer, an electron injection layer, and an electron transport layer may be provided between the first electrode 61 and the second electrode 62.

In a region of the stack of wiring layers 50 which overlaps the organic EL light emitting element 120 on the -Z side thereof, conductive plugs 53 and conductive layers 51 form wirings that are electrically coupled to the first electrode 61 and the second electrode 62 and extend to the inter-element wiring regions 80 and 90.

As shown in FIG. 7, the capacitance detecting portion 150 includes a first detecting electrode 152 and a second detecting electrode 153 facing each other in the Z direction and a capacitance portion 154 between the first detecting electrode 152 and the second detecting electrode 153. The first detecting electrode 152 is formed at the surface of the stack of wiring layers 50. The first detecting electrode 152 is a transparent electrode made of, for example, indium tin oxide (ITO). The capacitance portion 154 is made of an insulating material such as an acrylic resin. The second detecting electrode 153 is a light-transmitting electrode made of, for example, a silver-magnesium alloy (AgMg).

In a region of the stack of wiring layers 50 which overlaps the capacitance detecting portion 150 on the -Z side thereof, conductive plugs 53 and conductive layers 51 form wirings that are electrically coupled to the first detecting electrode 152 and the second detecting electrode 153.

As shown in FIGS. 6 and 7, the +Z-side surfaces of the organic EL light emitting element 120 and the capacitance detecting portion 150 are covered with a sealing layer 30. The sealing layer 30 is made of, for example, a light-transmitting inorganic material such as silicon dioxide (SiO₂) or silicon oxynitride (SiON). The sealing layer 30 may contain other materials to the extent that the sealing performance is not degraded. In the present embodiment, the sealing layer 30 is formed not only over the light emitting portion 12 and the contact detecting portion 15 but also over the light receiving portion 11 and the inter-element wiring regions 80 and 90. In the light receiving portion 11 and the inter-element wiring regions 80 and 90, the sealing layer 30 is formed at the surface of the stack of wiring layers 50.

The sealing layer 30 is covered with a cover plate 72 via a transparent resin layer 71. A surface of the cover plate 72 on the +Z side forms the detecting surface 10. The cover plate 72 is optically transparent and can be made of, for example, a glass plate or a quartz plate. The transparent resin layer 71 is made of, for example, a transparent resin such as an epoxy resin or an acrylic resin.

### Operation of Detecting Element

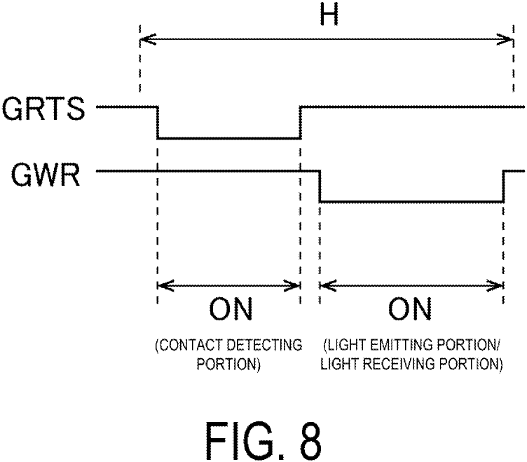

FIG. 8 is a timing chart of one detection cycle H of a detecting element P. In FIG. 8, GWR is a control signal supplied from the first scan line 81 and GRTS is a control signal supplied from the second scan line 82. The detecting device 3 repeats the detection cycle H for each detecting element P at predetermined time intervals. For example, the detection cycle H is repeated once every 1/16th of a second.

In the first half of the detection cycle H, the contact detecting portion 15 is switched on once as shown in FIG. 8. Specifically, a control signal is supplied from the second scan line drive circuit 25 to the second scan lines 82 for a predetermined period (GRTS: ON). As a result, the second switching transistor 151 is switched on for the predetermined period and the contact detection signal of the capacitance detecting portion 150 is output through the second signal line 92 for the predetermined period. The detecting device 3 determines whether it is in contact with the living body based on a change in the time constant of the contact detection signal corresponding to a change in the capacitance.

Subsequently, in the second half of the detection cycle H, on/off of the light emitting portion 12 and the light receiving portion 11 is controlled based on the immediately preceding contact detection signal. FIG. 8 shows an example of a detection cycle H when contact with the living body has been detected. In the present embodiment, the first switching transistor 121 and the third switching transistor 111 are configured to be switched on and off based on the output of the contact detecting portion 15. When it has been determined from the immediately preceding contact detection signal that contact with the living body has been detected, a control signal is supplied from the first scan line drive circuit 24 to the first scan line 81 for a predetermined period as shown in FIG. 8 (GWR: ON). Thereby, the first switching transistor 121 and the third switching transistor 111 are each switched on for the predetermined period, such that a drive current is supplied to the light emitting portion 12 to cause it to emit light and a detection signal corresponding to the intensity of light received by the light receiving portion 11 is output through the third signal line 93. Thus, biological information is detected.

In each detection cycle H, when contact with the living body has not been detected, no control signal is supplied to the first scan line 81 (GWR: OFF) unlike the timing chart of FIG. 8. Thus, when contact with the living body has not been detected, the light emitting portion 12 and the light receiving portion 11 are in an off state and biological information is not detected.

The detecting device 3 repeats the detection cycle H for each of the plurality of detecting elements P arranged in a matrix on the detecting surface 10. For example, the detection cycle H is performed for all detecting elements P. Alternatively, the detection cycle H is performed by sequentially selecting the detecting elements P one by one or in groups of a predetermined number.

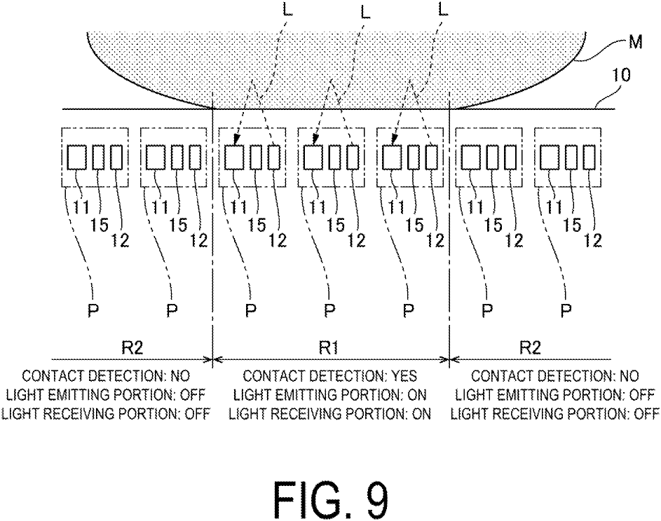

FIG. 9 is a diagram schematically showing an operating state of the detecting device 3. In the situation shown in FIG. 9, a central portion of the detecting surface 10 is in contact with the living body (the measuring site M) and a peripheral portion of the detecting surface 10 is not in contact with the living body (the measuring site M). In this case, the light emitting portion 12 and the light receiving portion 11 of each detecting element P in a contact region R1 that is in contact with the living body (the measuring site M) are switched on based on the detection of contact with the living body by the contact detecting portion 15 to detect biological information. On the other hand, the light emitting portion 12 and the light receiving portion 11 of each detecting element P in a noncontact region R2 that is not in contact with the living body (the measuring site M) remain off rather than being switched on because the contact detecting portion 15 has not detected contact with the living body. Thus, biological information is not detected.

### Main Functions and Advantages of Present Embodiment

As described above, the detecting device 3 of the present embodiment includes the substrate 20 and a plurality of detecting elements P arranged in a matrix at the substrate 20. Each of the plurality of detecting elements P includes a light emitting portion 12 that emits light toward the living body (the measuring site M), a light receiving portion 11 that can receive light incident from the living body based on the light emitted from the light emitting portion 12, and a contact detecting portion 15 that detects contact with the living body. The light emitting portion 12 in each of the plurality of detecting elements P emits light when the contact detecting portion 15 in the same detecting element P has detected contact with the living body.

The measuring apparatus 100 of the present embodiment includes the detecting device 3 and the control device 5 as an information analysis unit that identifies biological information from a detection signal indicating the detection result of the detecting device 3.

In the present embodiment, a plurality of detecting elements P arranged in a matrix on the detecting surface 10 are provided, contact with the living body is determined by each detecting element P, and the light emitting portion 12 in the same detecting element is switched on and off based on the contact determination. In addition, the light receiving portion 11 in the same detecting element can be switched on and off in synchronization with the light emitting portion 12. By doing so, even if the contact state between the detecting surface 10 and the living body is partially bad, measurement can be performed by selecting detecting elements P that are in good contact with the living body rather than switching the entirety of the detecting device 3 off due to bad wearing. Thus, it is possible to reduce situations where it is determined that wearing is bad and measurement is not possible. As a result, wearing discomfort and burden can be reduced because there is no need to tighten a fastener for wearing to prevent the occurrence of bad wearing. Moreover, measurement can be performed with less power consumption because detecting elements P with poor contact are switched off. Thus, power saving can be achieved. Furthermore, it is possible to prevent deterioration in signal accuracy due to disturbance light or stray light because detecting elements P that are in good contact with the living body can be selected to perform measurement.

In the present embodiment, the light emitting portion 12 of each detecting element P includes a first switching transistor 121 as a first switching element that is switched on and off based on the output of the contact detecting portion 15 in the same detecting element. Thus, when on/off of the light emitting portion 12 of each detecting element P is controlled, it is possible to actively drive the light emitting portion 12.

In the present embodiment, the substrate 20 on which each detecting element P is formed is made of a semiconductor material. The light emitting portion 12 includes a top emission type organic EL light emitting element 120 formed at the substrate 20. Employing the top-emission type light emitting element structure can increase the amount of emitted light, such that a necessary amount of light can be secured even if the amount of power supplied is reduced, decreasing the amount of emitted light. Thus, power consumption of the light emitting portion 12 can be reduced and power saving can be achieved.

In the present embodiment, the light receiving portion 11 includes a photodiode 110 formed at the substrate 20. Further, the contact detecting portion 15 outputs a contact detection signal corresponding to a change in the capacitance between the first detecting electrode 152 and the second detecting electrode 153 formed at the substrate 20. By forming the light emitting element, the light receiving element, and the contact detecting element using a semiconductor substrate as a base in this way, the size and thickness of the detecting element P can be reduced compared to when elements implemented as chips are mounted on a substrate as in the related art. Also, a plurality of detecting elements P can be arranged close to each other on the same substrate 20. Thus, the size and thickness of the detecting device 3 can be reduced.

For example, when an LED and a photodiode implemented as chips are mounted on a substrate, the gap between the light receiving portion 11 and the light emitting portion 12 is in millimeters, but in the configuration of the present embodiment, the gap between the light receiving portion 11 and the light emitting portion 12 is in microns. Similarly, the gap between the contact detecting portion 15 and the light receiving and emitting portions 11 and 12 is in microns. Thus, the size of the detecting device 3 can be reduced even when the detecting device 3 is constructed by arranging a plurality of detecting elements P in a matrix.

When the light emitting portion 12 and the light receiving portion 11 are close to each other, the amount of received light, which has been returned to the light receiving portion 11 from the measuring site M after being emitted from the light emitting portion 12, is large. Thus, even if the amount of light emitted from the light emitting portion 12 is reduced, a necessary amount of received light can be secured, such that power consumption of the light emitting portion 12 can be reduced. Accordingly, power saving of the detecting device 3 can be achieved. In addition, the S/N ratio can be increased because a large amount of light returning from the measuring site M can be received and noise is small. Further, because noise is small, the light receiving portion 11 does not need an angle limiting filter or a bandpass filter, such that the thickness of the detecting element P can be reduced.

In particular, when the light L emitted from the light emitting portion 12 is green light, the green light returns after diffusing only to a shallow region in the body of the subject, such that the amount of received light rapidly decreases away from the light emitting portion 12. Thus, by bringing the light receiving portion 11 and the light emitting portion 12 close to each other, it is possible to increase the amount of received light when the light L emitted from the light emitting portion 12 is green light. Accordingly, power saving can be achieved and the S/N ratio can be increased.

In the present embodiment, a first scan line 81 extending in the X direction (a first direction) and a first signal line 91, a second signal line 92, and a third signal line 93 extending in the Y direction (a second direction) intersecting the X direction are formed at the substrate 20. The light emitting portion 12 is controlled via a first switching transistor 121 coupled to the first scan line 81 and the first signal line 91. The contact detecting portion 15 outputs a contact detection signal via the second signal line 92. The light receiving portion 11 outputs a detection signal of the light emitted from the living body via the third signal line 93. Thus, the detecting device 3 can have a structure that uses the pixel structure of an organic EL display panel. The light emitting portion 12 of each detecting element P can be driven using the pixel driving method of an organic EL display panel. Further, the detection signal from the light receiving portion 11 and the contact detection signal from the contact detecting portion 15 can be output using the same wiring structure as that with scan lines and signal lines for driving pixels.

In the present embodiment, a second scan line 82 extending in the X direction (the first direction) is also formed at the substrate 20. The contact detecting portion 15 includes a second switching transistor 151 as a second switching element coupled to the second scan line 82. The second switching transistor 151 controls the output of the contact detection signal via the second signal line 92. Because the contact detecting portion 15 includes such a switching element, the contact detecting portion 15 can be actively driven when each detecting element P detects contact with the living body.

Further, in the present embodiment, the light receiving portion 11 includes a third switching transistor 111 as a third switching element that is switched on and off based on the output of the contact detecting portion 15 in the same detecting element. The third switching transistor 111 is coupled to the first scan line 81 and controls the output of the detection signal via the third signal line 93. Because the light receiving portion 11 includes such a switching element, the light receiving portion 11 can be actively driven when the light emitting portion 12 and the light receiving portion 11 are turned on and off in each detecting element P. Also, because the switching element of the light emitting portion 12 and the switching element of the light receiving portion 11 are coupled to a common scan line (the first scan line 81), the light emitting portion 12 and the light receiving portion 11 can be synchronously driven by a common control signal.

### Modifications

(1) The circuit configuration of the detecting element P is not limited to the configuration shown in FIG. 5. For example, while the light emitting portion 12 and the light receiving portion 11 are coupled to a common scan line (the first scan line 81) in the configuration shown in FIG. 5, it is possible to employ a configuration in which the light emitting portion 12 and the light receiving portion 11 are coupled to and controlled via different scan lines.

(2) While both the light emitting portion 12 and the light receiving portion 11 are actively driven in the configuration shown in FIG. 5, it is possible to employ a configuration in which one or both of the light emitting portion 12 and the light receiving portion 11 are passively driven. It is possible to employ a configuration in which the contact detecting portion 15 is also passively driven.

(3) The shape and arrangement of the light receiving portion 11, the light emitting portion 12, and the contact detecting portion 15 in each detecting element P are not limited to the shape and arrangement shown in FIG. 4. For example, a light receiving portion 11, a light emitting portion 12, and a contact detecting portion 15 may be arranged in a line in the X direction or the Y direction. Alternatively, a configuration in which a light receiving portion 11 is arranged to surround a light emitting portion 12 may be adopted or a configuration in which a contact detecting portion 15 is arranged to surround a light receiving portion 11 and a light emitting portion 12 may be adopted.

(4) The plurality of detecting elements P may include those that emit light in different wavelength bands. For example, the plurality of detecting elements P may include a first detecting element that emits first light through a light emitting portion 12, a second detecting element that emits second light through a light emitting portion 12, and a third detecting element that emits third light through a light emitting portion 12. The first light is, for example, green light having a green wavelength band of 520 nm to 550 nm and a peak wavelength of 520 nm. The second light is, for example, red light having a red wavelength band of 600 nm to 800 nm and a peak wavelength of 660 nm. The third light is, for example, near-infrared light having a near-infrared wavelength band of 800 nm to 1300 nm and a peak wavelength of 905 nm. In this case, it is possible to identify the pulse rate of the subject by identifying the heartbeat of the subject from a detection signal representing the received light intensity of the first light (green light). Additionally, it is possible to identify the oxygen saturation (SpO₂) of the subject by analyzing a detection signal representing the received light intensity of the second light (red light) and a detection signal representing the received light intensity of the third light (near-infrared light).

What is claimed is:

1. A detecting device comprising a substrate and a plurality of detecting elements arranged in a matrix at the substrate,
wherein each of the plurality of detecting elements includes a light emitting portion configured to emit light toward a living body, a light receiving portion configured to receive light incident from the living body based on the light emitted from the light emitting portion, and a contact detecting portion configured to detect a contact with the living body, and
the light emitting portion in each of the plurality of detecting elements is configured to emit light based on the detection of the contact with the living body by the contact detecting portion in the same detecting element.

2. The detecting device according to claim 1, wherein the light emitting portion includes a first switching element configured to be switched on and off based on an output of the contact detecting portion in the same detecting element.

3. The detecting device according to claim 2, wherein the substrate is made of a semiconductor material, and
the light emitting portion includes a top emission type organic electroluminescent light emitting element formed at the substrate.

4. The detecting device according to claim 3, wherein the contact detecting portion is configured to output a contact detection signal corresponding to a change in a capacitance between a first detecting electrode and a second detecting electrode formed at the substrate.

5. The detecting device according to claim 4, wherein the light receiving portion includes a photodiode formed at the substrate.

6. The detecting device according to claim 5, wherein a first scan line extending in a first direction and a first signal line, a second signal line, and a third signal line extending in a second direction intersecting the first direction are formed at the substrate,
the light emitting portion is configured to be controlled via the first switching element coupled to the first scan line and the first signal line,
the contact detecting portion is configured to output the contact detection signal via the second signal line, and
the light receiving portion is configured to output a detection signal of the light emitted from the living body via the third signal line.

7. The detecting device according to claim 6, wherein a second scan line extending in the first direction is formed at the substrate,
the contact detecting portion includes a second switching element coupled to the second scan line, and
the second switching element is configured to control the output of the contact detection signal via the second signal line.

8. The detecting device according to claim 6, wherein the light receiving portion includes a third switching element configured to be switched on and off based on the output of the contact detecting portion in the same detecting element, and
the third switching element is coupled to the first scan line and configured to control the output of the detection signal via the third signal line.

9. A measuring apparatus comprising:
the detecting device according to claim 1; and
an information analysis unit configured to identify biological information from a detection signal indicating a detection result of the detecting device.
